(19) United States
(12) Patent Application Publication
(10) Pub. No.: US 2015/0164352 A1
(43) Pub. Date: Jun. 18, 2015

(54) APPARATUS FOR MEASURING BIO-INFORMATION AND A METHOD FOR ERROR COMPENSATION THEREOF

(71) Applicant: LG ELECTRONICS INC., Seoul (KR)

(72) Inventors: Hyoung Kil YOON, Seoul (KR); Inah JO, Seoul (KR); Wonhyeog JIN, Seoul (KR)

(73) Assignee: LG ELECTRONICS INC., Seoul (KR)

(21) Appl. No.: 14/466,693

(22) Filed: Aug. 22, 2014

(30) Foreign Application Priority Data
Dec. 18, 2013 (KR) 10-2013-0158169
Dec. 24, 2013 (KR) 10-2013-0162325

(51) Int. Cl. A61B 5/024; A61B 5/00; A61B 5/11

(52) U.S. Cl. CPC A61B 5/02427; A61B 5/1102; A61B 5/6824; A61B 5/681

(57) ABSTRACT

The present disclosure relates to an apparatus for measuring bio-information, the apparatus including a heart rate sensor unit configured to measure heart rates by receiving a light that has entered and come out from skin, an acceleration sensor unit configured to output a step count by measuring the step count of a wearer, a display unit configured to display the measured heart rates and step count, and a wrist-wearable connection unit configured to electrically connect the heart rate sensor, the acceleration sensor unit and the display unit, whereby heart rate can be accurately measured using a line light source instead of point light source, even if the apparatus is not fully attached to a wrist. The apparatus includes a heart rate sensor unit configured to improve a light receiving efficiency using four light receiving units.

### CROSS-REFERENCE TO RELATED APPLICATIONS

Pursuant to 35 U.S.C. §119(a), this application claims the benefit of earlier filing date and right of priority to Korean Patent Application Nos. 10-2013-0158169, filed on Dec. 18, 2013, and 10-2013-0162325, filed on Dec. 24, 2013, the contents of which are all hereby incorporated by reference herein in their entirety.

### BACKGROUND OF THE INVENTION

1. Field of the Invention

The present disclosure relates to an apparatus for measuring bio-information and a method for error compensation thereof.

2. Description of Related Art

A conventional wearable activity tracker may be classified into two types. That is, a band type product made of elastic materials such as band type silicone or rubber, and a wristwatch type product. The former is wrist-unattachable and equipped only with a pedometer function mounted with an acceleration sensor, while the latter is equipped with an optical heart rate sensor to provide health information of a wearer such as heart rate activity state in addition to pedometer function.

Meanwhile, the optical pulse sensor estimates heart rate activities by measuring blood flowing in the blood vessel using an optical characteristic of bio tissues. To be more specific, PPG (photoplethysmogram) observes optical characteristics of bio tissues such as light reflectivity, absorptance and transmittance that show during volumetric change in blood vessel by using a light, and measures the heart rate using the change. The method of non-invasive method is widely used due to enablement of measuring bio signals, advantageous because of miniaturized size and convenience in usage, and conducive to development of wearable life signal detection sensor.

The optical heart rate sensor includes a light source and a light receiver, where when a light is incident on skin from the light source, and a light reflected from the incident light is collected by the light receiver, the number of heart rates can be detected from changes in quantity of the collected light.

However, the conventional optical heart rate sensor is disadvantageous in that only one PH (Photo diode) is available, a light source takes a shape of a point light source, and heart rate can be measured by being attached to skin. Thus, the conventional optical heart rate sensor is applicable only to a wrist watch type product, and is relatively difficult to be applied to a wrist-unattachable, band type product.

### SUMMARY OF THE INVENTION

Exemplary aspects of the present disclosure are to substantially solve at least the above problems and/or disadvantages and to provide at least the advantages as mentioned below.

Thus, the present disclosure is directed to provide an apparatus for measuring bio-information and a method for error compensation therefor.

In one general aspect of the present invention, there is provided an apparatus for measuring bio-information, the apparatus comprising:

a heart rate sensor unit configured to measure heart rates by receiving a light that has entered and come out from skin;

an acceleration sensor unit configured to output a step count by measuring the step count of a wearer;

a display unit configured to display the measured heart rates and step count; and

a wrist-wearable connection unit configured to electrically connect the heart rate sensor, the acceleration sensor unit and the display unit.

Preferably, but not necessarily, the heart rate sensor unit may include,

a light source unit configured to emit a linear light source,

a light receiving unit configured to receive the light that has entered and come out from the skin from the light emitted from the light source, and

a controller configured to detect the heart rates from a quantity of light received by the light receiving unit.

Preferably, but not necessarily, the light source unit may include,

an LED (Light Emitting Diode) configured to emit a light of point light source,

a curved light guide of a particular curvature radius configured to advance the emitted light to a particular direction,

a plurality of V-shaped patterns configured to emit a light to a particular direction by refracting the emitted light, and

a reflective plate configured to reflect a light emitted to an outside from the light guide into an interior of the light guide.

Preferably, but not necessarily, the LED may use a yellowish green color light source.

Preferably, but not necessarily, the light receiving unit may include,

a first light receiving unit arranged at an upper left surface based on a lengthwise direction of the light source,

a second light receiving unit arranged at an upper right surface based on a lengthwise direction of the light source,

a third light receiving unit arranged at a bottom left surface based on a lengthwise direction of the light source, and

a fourth light receiving unit arranged at a bottom right surface based on a lengthwise direction of the light source.

Preferably, but not necessarily, the light receiving unit may include,

a photodetector configured to receive a light that has entered and come out of skin from a light emitted from the light source unit, and

a light receiving house configured to wrap the photodetector and to gradually broaden at an entrance toward a skin contact surface.

Preferably, but not necessarily, the plurality of V-shaped patterns may taper off at a spacing of adjacent patterns as being distanced from the LED.

Preferably, but not necessarily, the connection unit may be a wrist band type connector unit of elastic material.

Preferably, but not necessarily, the connection unit may be a wrist watch type, wrist-attachable connector unit.

In another general aspect of the present disclosure, there is provided a method for error compensation in a heart rate sensor including a first light receiving unit arranged at an upper left surface of a line light source, a second light receiving unit arranged at an upper right surface of the line light source,

a third light receiving unit arranged at a bottom left surface of the line light source, and a fourth light receiving unit arranged at a bottom right surface of the line light source, the method comprising:

detecting an error by comparing light signals received by the first to fourth light receiving units;

multiplying a predetermined weight to a greater light signal as a result of comparison of the light signals when the error is generated; and

adding a size of a light signal multiplied by the weight to a size of a light signal not multiplied by the weight.

Preferably, but not necessarily, the error may include a first error, which is a difference between a first signal, which is a sum of light signals received by the first and second light receiving units and a second signal, which is a sum of light signals received by the third and fourth light receiving units,

a second error, which is a difference between a third signal, which is a sum of light signals received by the first and third light receiving units and a fourth signal, which is a sum of light signals received by the second and fourth light receiving units, and

a third error, which is a difference between a fifth signal, which is a sum of light signals received by the first and fourth light receiving units and a sixth signal, which is a sum of light signals received by the second and third light receiving units.

Preferably, but not necessarily, the step of multiplying a predetermined weight to a greater light signal as a result of comparison of the light signals when the error is generated may include,

multiplying a predetermined weight to a greater signal between the first and second signals when the first error is generated,

multiplying a predetermined weight to a greater signal between the third and fourth signals when the second error is generated, and

multiplying a predetermined weight to a greater signal between the fifth and sixth signals when the first error is generated.

Preferably, but not necessarily, the method may further comprise determining a greater value as a heart rate reference signal between a size of an optical signal multiplied by the weight and a size of an optical signal not multiplied by the weight.

In still another general aspect of the present disclosure, there is provided a method for error compensation in a heart rate sensor including a first light receiving unit arranged at an upper left surface of a line light source, a second light receiving unit arranged at an upper right surface of the line light source,

a third light receiving unit arranged at a bottom left surface of the line light source, and a fourth light receiving unit arranged at a bottom right surface of the line light source, the method comprising:

detecting an error by comparing light signals received by the first to fourth light receiving units;

multiplying a predetermined weight to a greater light signal as a result of comparison of the light signals when the error is generated; and

adding a size of a light signal multiplied by the weight to a size of a light signal not multiplied by the weight.

Preferably, but not necessarily, the step of detecting an error by comparing light signals received by the first to fourth light receiving units may include determining a light receiving unit having received a largest size of optical signal by comparing a first error, which is a difference between a first signal, which is a sum of light signals received by the first and second light receiving units and a second signal, which is a sum of light signals received by the third and fourth light receiving units, a second error, which is a difference between a third signal, which is a sum of light signals received by the first and third light receiving units and a fourth signal, which is a sum of light signals received by the second and fourth light receiving units, and a third error, which is a difference between a fifth signal, which is a sum of light signals received by the first and fourth light receiving units and a sixth signal, which is a sum of light signals received by the second and third light receiving units.

### ADVANTAGEOUS EFFECTS

The present disclosure has an advantageous effect in that heart rate can be accurately measured using a line light source instead of point light source, even if the apparatus for measuring the bio-information is not fully attached to a wrist.

Another advantageous effect is that the light receiving efficiency can be greatly enhanced using four light receiving units.

Still another advantageous effect is that the present invention can be applied to both the wrist band type connector unit and a wrist watch typed connector unit.

Still further advantageous effect is that a heart rate signal can be stably detected even if a heart rate sensor including a plurality of light receiving units is not attached to skin.

Still further advantageous effect is that the present invention can choose an appropriate method between a comparative signal weighting method and an absolute signal weighting method in consideration of weight setting, SNR (Signal-to-Noise-Ratio) and operation environment of heart rate sensor.

### BRIEF DESCRIPTION OF THE DRAWINGS

FIG. 1 is a block diagram illustrating a configuration of an apparatus for measuring bio-information according to an exemplary embodiment of the present disclosure.

FIG. 2 is a block diagram illustrating a configuration of a heart rate sensor unit according to an exemplary embodiment of the present disclosure.

FIG. 3 is a schematic view illustrating arrangement of a light source unit of a heart rate sensor unit, and first to fourth light receiving units according to an exemplary embodiment of the present disclosure.

FIG. 4 is a schematic view illustrating a structure of a light source unit according to an exemplary embodiment of the present disclosure.

FIGS. 5 and 6 are respectively a perspective view and a bottom view of a structure of a light source unit according to an exemplary embodiment of the present disclosure.

FIG. 7 is a plan view illustrating structures of light source unit of heart rate sensor unit, and first to fourth light receiving units according to an exemplary embodiment of the present disclosure.

FIG. 8 is a lateral view illustrating structures of first to fourth light receiving units according to an exemplary embodiment of the present disclosure.

FIG. 9 is a schematic view illustrating a heart rate sensor unit attached to skin of an apparatus for measuring bio-information according to an exemplary embodiment of the present disclosure.

FIG. 10 is a schematic view illustrating a structure of an apparatus for measuring bio-information according to an exemplary embodiment of the present disclosure.

FIG. 11 is a schematic view illustrating types of alignment errors to be considered when a method for error compensation according to an exemplary embodiment of the present disclosure is performed.

FIG. 12 is a schematic view illustrating a method for error compensation according to an exemplary embodiment of the present disclosure.

FIG. 13 is a schematic view illustrating a comparative signal weighting method in a method for error compensation according to an exemplary embodiment of the present disclosure.

FIG. 14 is a schematic view illustrating an absolute signal weighting method in a method for error compensation according to an exemplary embodiment of the present disclosure.

### DETAILED DESCRIPTION OF THE INVENTION

Various exemplary embodiments will be described more fully hereinafter with reference to the accompanying drawings, in which some exemplary embodiments are shown. The present inventive concept may, however, be embodied in many different forms and should not be construed as limited to the example embodiments set forth herein. Rather, the described aspect is intended to embrace all such alterations, modifications, and variations that fall within the scope and novel idea of the present disclosure.

Now, an exemplary embodiment of the present disclosure will be described in detail with reference to the accompanying drawings.

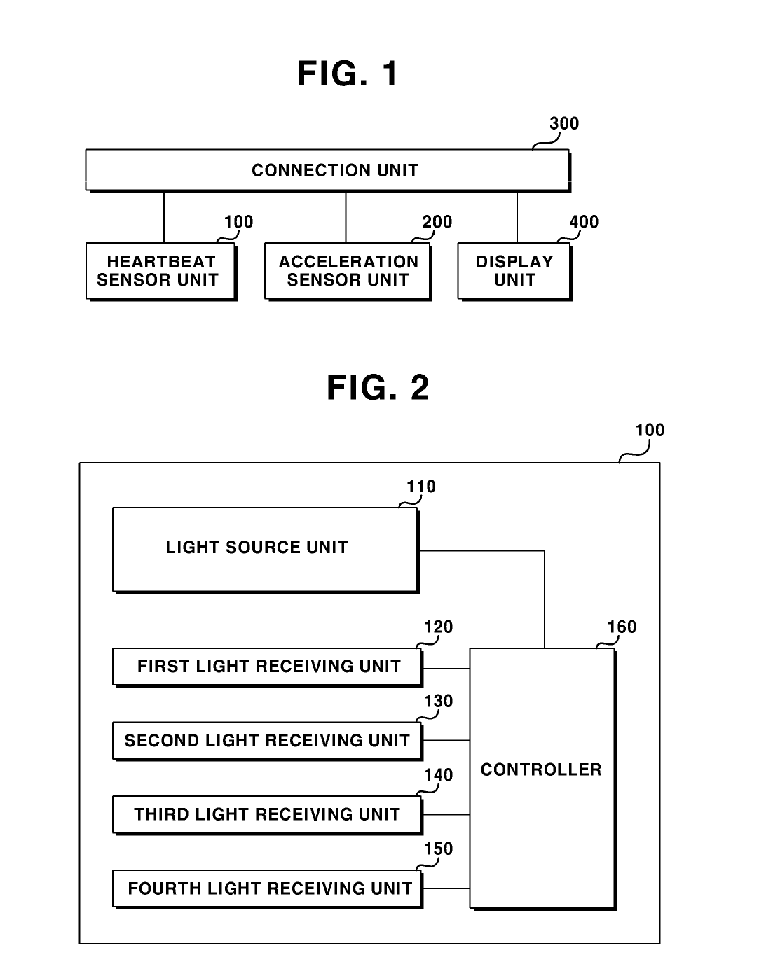

FIG. 1 is a block diagram illustrating a configuration of an apparatus for measuring bio-information according to an exemplary embodiment of the present disclosure.

Referring to FIG. 1, an apparatus for measuring bio-information (hereinafter referred to as “apparatus”) may include a heart rate sensor unit (100), an acceleration sensor unit (200), a wrist-wearable connection unit (300) and a display unit (400).

The heart rate sensor unit (100) may include an optical heart rate sensor to display a heart rate (pulse) of a wearer on the display unit (400) by measuring the heart rate of the wearer. The heart rate sensor unit (100) uses a principle, in which a light that has entered and come out from the skin from the light emitted from a light source is changed by heart rate in time in terms of optical absorption degree in response to plastid in blood such as hemoglobin available in skin tissue and blood, where the heart rate sensor unit (100) receives a light returned by the light receiving unit of the heart rate sensor unit (100) and detects the heart rate by converting the received light to an electrical signal.

The acceleration sensor unit (200) may measure an acceleration of an apparatus for measuring bio-information (apparatus) according to an exemplary embodiment of the present disclosure and provide a pedometer function. The acceleration sensor unit (200) may include an acceleration sensor mounted on a pedometer or a walk-step measuring device, measure an acceleration speed in response to movement of a wearer of the apparatus, and display on the display unit (400) a step count by converting the measured acceleration speed to the step count.

The connection unit (300) functions to connect the heart rate sensor unit (100), the acceleration sensor unit (200) and the display unit (400), and may take a shape of being wearable on a wrist, a fore arm, an upper arm, a thigh, a head and/or a finger. The connection unit (300) may be formed in the shape of a band type connection unit of elastic material such as silicone and a rubber, may be formed in the shape of a wrist watch type connection unit wearable to be attached to a wrist like an wrist watch, or may be formed in the shape of a head band type connection unit wearable to be attached to a head like a head band.

The display unit (400) may display heart rate information measured by the heart rate sensor unit (100). Furthermore, the display unit (400) may display pedometer information measured by the acceleration sensor unit (200).

FIG. 2 is a block diagram illustrating a configuration of a heart rate sensor unit (100) according to an exemplary embodiment of the present disclosure.

Referring to FIG. 2, the heart rate sensor unit (100) according to an exemplary embodiment of the present disclosure may include a light source unit (110), first to fourth light receiving units (120, 130, 140, 150) and a controller (160).

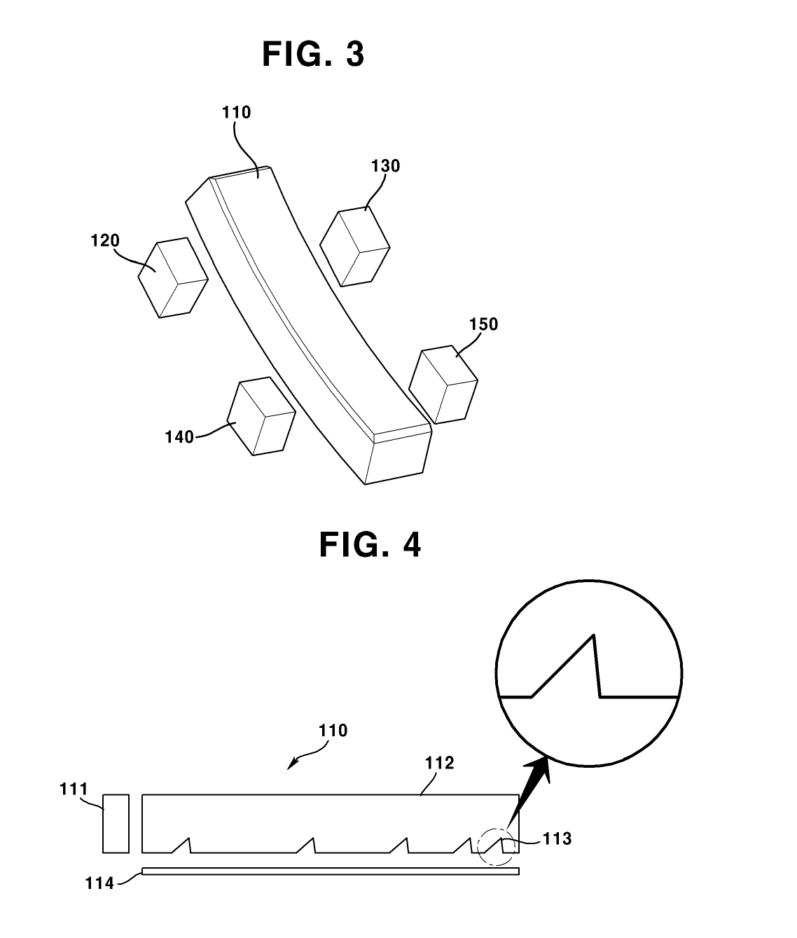

The light source unit (110) emits a light. The light source unit (110) may use an optical light guide for emitting a line light source. The use of line light source may irradiate a light on a broader skin area than that of point light source, whereby a sufficient quantity of light can be incident on a wrist area even if the apparatus is not closely attached to the wrist. A detailed structure of the light source unit (110) will be described with reference to FIGS. 3 to 6 later.

The first to fourth light receiving units (120, 130, 140, 150) may be symmetrically (vertically and horizontally) arranged based on lengthwise direction of the light source unit (110), and may receive a light emitted from the light source unit (110) that has entered and come out from the skin. The arrangement of the first to fourth light receiving units (120, 130, 140, 150) may be changed depending on a length and an area of the light source unit (110).

The first to fourth light receiving units (120, 130, 140, 150) may include first to fourth photodetectors (121, 131, 141, 151), and the first to fourth photodetectors (121, 131, 141, 151) may be PDs (Photo Diodes). The first to fourth light receiving units (120, 130, 140, 150) may transmit the received light to the controller (160) by converting the received light to an electrical signal.

The controller (160) may detect quantity of light emitted from the light source unit (110) and may receive the light received by the first to fourth light receiving units (120, 130, 140, 150) in electrical signal.

FIG. 3 is a schematic view illustrating arrangement of the light source unit (110) of the heart rate sensor unit (100), and the first to fourth light receiving units (120, 130, 140, 150) according to an exemplary embodiment of the present disclosure.

Referring to FIG. 3, the light source unit (110) may be a line light source unit configured to take a shape of a curved surface. When a curved line light source is employed, the light source unit can be attached to a measurement unit having a curve as in the wrist. The first light receiving unit (120) may be arranged at an upper left surface based on a lengthwise direction of the light source, the second light receiving unit (130) may be arranged at an upper right surface based on a lengthwise direction of the light source, the third light receiving unit (140) may be arranged at a bottom left surface based on a lengthwise direction of the light source, and the fourth light receiving unit (150) may be arranged at a bottom right surface based on a lengthwise direction of the light source.

FIG. 4 is a schematic view illustrating a structure of the light source unit (110) according to an exemplary embodiment of the present disclosure.

Referring to FIG. 4, although the light source unit (110) may be preferably a line light source unit configured to take a shape of a curved surface, the light source unit is illustrated herein as a line light source unit having no curvature, for convenience sake.

The light source unit may include an LED (Light Emitting Diode, 111), a curved light guide (112), a plurality of V-shaped patterns (113) and a reflective plate (114).

The LED (111) may emit a light of point light source shape to a lengthwise direction of the light source unit (110). Although only one LED (111) is illustrated at a distal end in FIG. 4D, a total of two LEDs may be arranged each at a distal end.

Although the LED (111) uses a wavelength of red or an infrared region, the LED (111) preferably uses a yellowish green color light source in consideration of optical characteristic of skin tissue. The curved light guide (112) is preferred to have a particular curvature radius of about 20 mm to allow being attached to a radial artery of the wrist when the heart rate sensor unit (100) is brought into contact with the wrist. The curved light guide (112) may be formed with a flexible material.

The plurality of V-shaped patterns (113) may taper off at a spacing of adjacent patterns as being distanced from the LED (111), which is to emit as much as uniform refracted light because the quantity of reached light decreases as being distanced from the light source.

The reflective plate (114) may be arranged on all surfaces except for a direction from which the light is emitted from the curved light guide (112) to reflect the light into an interior of the light guide, whereby the light loss can be minimized.

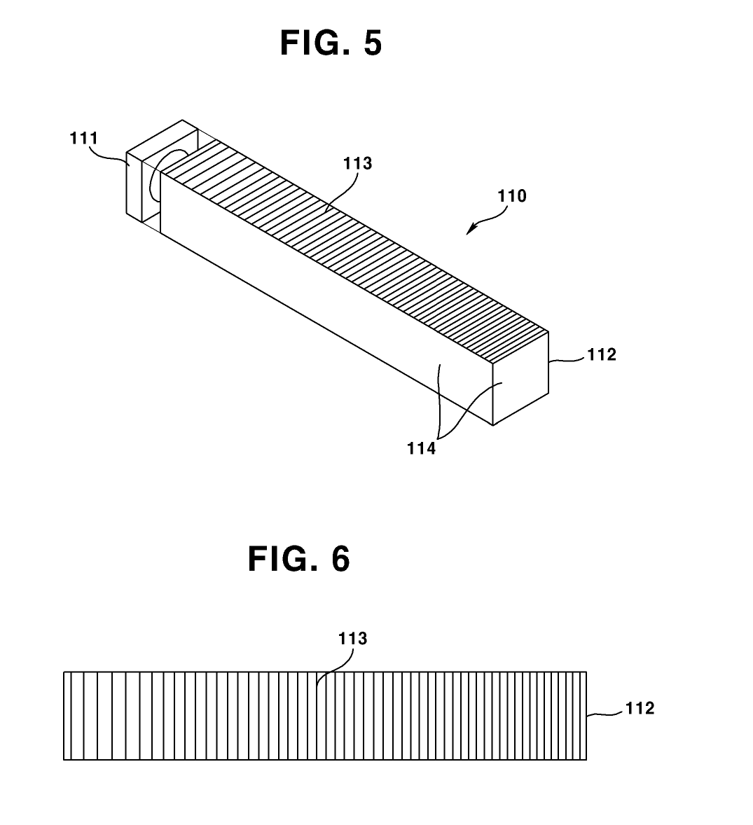

FIGS. 5 and 6 are respectively a perspective view and a bottom view of a structure of a light source unit according to an exemplary embodiment of the present disclosure.

Referring to FIGS. 5 and 6, although the light source unit (110) may be preferably a line light source unit configured to take a shape of a curved surface, the light source unit is illustrated herein as a line light source unit having no curvature, for convenience sake.

The plurality of V-shaped patterns (113) may taper off at a spacing of adjacent patterns as being distanced from the LED (111), which is to emit as much uniform refracted light as possible, because the quantity of reached light decreases as being distanced from the light source.

The curved light guide (112) is formed with a plurality of V-shaped patterns (113) to minimize the quantity of leaked light by arranging the reflective plate (114) on all surfaces except for a direction from which the light is emitted.

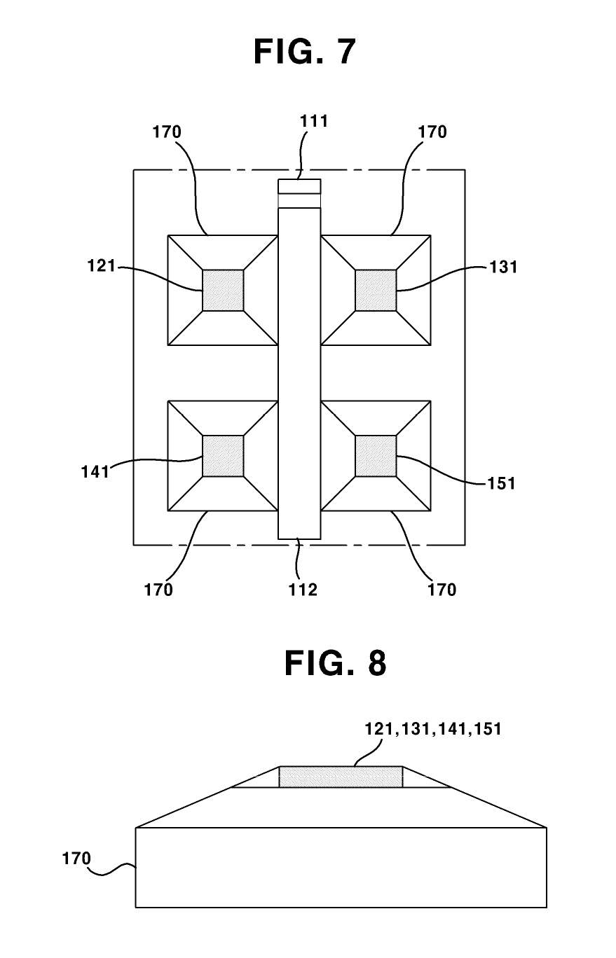

FIG. 7 is a plan view illustrating structures of light source unit (110) of heart rate sensor unit (100), and first to fourth light receiving units (120, 130, 140, 150) according to an exemplary embodiment of the present disclosure.

Referring to FIG. 7, the first to fourth light receiving units (120, 130, 140, 150) may be symmetrically (vertically and horizontally) arranged based on lengthwise direction of the light source unit (110).

Each of the first to fourth light receiving units (120, 130, 140, 150) may take a combined shape of first to fourth photodetectors (121, 131, 141, 151) and a light receiving housing (170). The first to fourth photodetectors (121, 131, 141, 151) may be PDs (Photo Diodes) configured to receive light emitted from the light source unit that has entered and come out of the skin.

The light receiving housing (170) may be surface-treated with a hollow material of high reflectivity to take a shape of wrapping the first to fourth photodetectors (121, 131, 141, 151). When the PD is directly arranged on the surface of skin using no light receiving housing, the light receiving rate of light that has entered and come out of the skin may decrease, such that the light receiving housing (170) may be employed to collect as much light as possible.

The PD may be positioned at an area inside the light receiving housing distanced at a predetermined space from the skin. The light receiving housing (170) may take a shape of a bottomless quadrangular pyramid.

FIG. 8 is a lateral view illustrating structures of first to fourth light receiving units (120, 130, 140, 150) according to an exemplary embodiment of the present disclosure.

Referring to FIG. 8, the first to fourth light receiving units (120, 130, 140, 150) may include the first to fourth photodetectors (121, 131, 141, 151) and the light receiving housing (170).

The light receiving housing (170) may have a hollow inside to prevent from being directly contact to the skin and take a shape of wrapping the first to fourth photodetectors (121, 131, 141, 151) to collect the light that has entered and come out from the skin.

Referring to FIG. 8, the light receiving housing (170) may take a shape of a hollow and bottomless quadrangular pyramid to include the first to fourth photodetectors (121, 131, 141, 151), the present disclosure is not limited thereto, and the light receiving housing (170) may take a shape of any configuration capable of collecting light and being attached to the skin, such as a circular truncated cone, a cube and/or a rectangular parallelepiped, each being less a bottom surface.

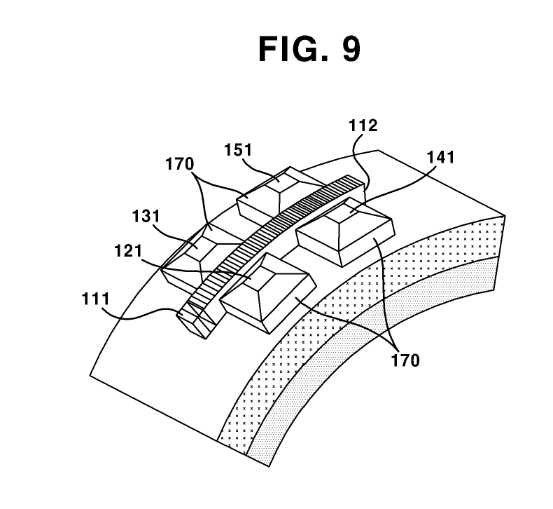

FIG. 9 is a schematic view illustrating a heart rate sensor unit (100) attached to skin of an apparatus for measuring bio-information according to an exemplary embodiment of the present disclosure.

Referring to FIG. 9, the light source unit (110) of the heart rate sensor unit (100) may include an LED (111) and a curved light guide (112).

The light source unit (110) may be centrally formed with the curved light guide (112) having a predetermined radius of curvature to be attached to a wrist area, and may symmetrically (vertically and horizontally) include the first to fourth light receiving units (120, 130, 140, 150) based on a lengthwise direction of the light source unit (110) to allow the LED to come thereon. Each of the first to fourth light receiving units (120, 130, 140, 150) may include the first to fourth photodetectors (121, 131, 141, 151), and a light receiving housing (170) of a bottomless quadrangular pyramid shape.

It is preferable that the heart rate sensor unit (100) be completely attached the skin, but if the heart rate sensor unit (100) is completely attached to the skin, the wearer may feel uncomfortable, such that the connection unit (300) may be in a band type connection unit of elastic material such as silicone and a rubber. Although the band type connection unit may be formed with a space between the band and the attached area with the skin, the heart rate sensor unit (100) according to the present disclosure can emit a sufficient quantity of light using a line light source and four light receiving units to allow measuring an accurate heart rate count.

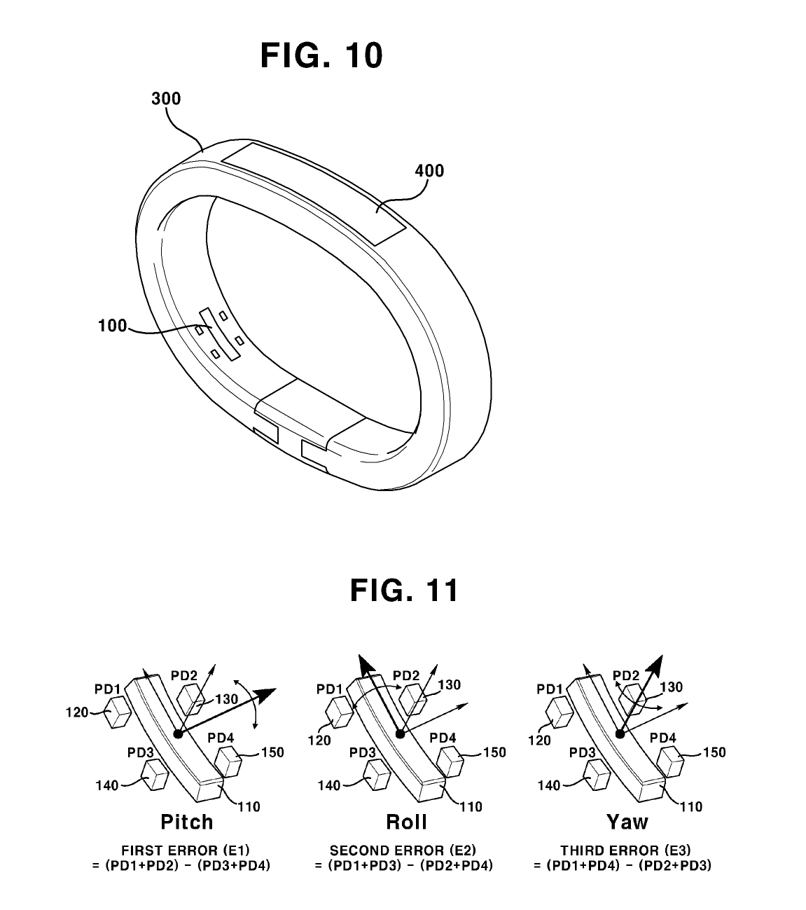

FIG. 10 is a schematic view illustrating a structure of an apparatus for measuring bio-information according to an exemplary embodiment of the present disclosure.

Referring to FIG. 10, the apparatus for measuring bio-information according to an exemplary embodiment of the present disclosure may include a heart rate sensor unit (100), a connection unit (300) and a display unit (400). As illustrated in FIG. 1, an acceleration sensor unit (200) may be additionally formed in the apparatus, and other bio-information measuring modules may be further added thereto.

Furthermore, the connection unit (300) may be designed to replace various modules used in the apparatus. Although the connection unit (300) in FIG. 10 is illustrated in the form of a band type connection unit, the present disclosure is not limited thereto, and may be formed in the shape of a wrist watch type connection unit wearable to be attached to a wrist like an wrist watch, and any type wearable to the wrist is also acceptable. The display unit (400) may display various bio-information in addition to a heart rate signal measured by the heart rate sensor unit (100). For example, when a temperature sensor module (not shown) is worn by a wearer, a body temperature may be measured and displayed on the display unit (400).

FIG. 11 is a schematic view illustrating types of alignment errors to be considered when a method for error compensation according to an exemplary embodiment of the present disclosure is performed.

In ideal case, that is, when the heart rate sensor unit is completely attached to the skin, a sum of quantities of lights incident on from each light receiving unit would have a predetermined value. To be more specific, when the heart rate sensor unit is completely attached to the skin, all the light receiving units and the light source unit come to touch the skin to allow a light, which is a subject of detection, to be incident on four light receiving units. Thus, a sum, in which all the quantities of lights incident on the light receiving units are added, may have a predetermined value.

However, when the heart rate sensor unit is not completely attached to the skin to allow some of the light receiving units to be attached to the skin and to allow some of the light receiving units not to be attached to the skin, there may be generated an alignment error. Referring to FIG. 11, quantity of light incident on the first to fourth light receiving units (120, 130, 140, 150) may be respectively defined as a first light receiving signal (PD1), a second light receiving signal (PD2), a third light receiving signal (PD3), and a fourth light receiving signal (PD4).

According to the method for error compensation according to an exemplary embodiment of the present disclosure, when check is made on a sum (PD1+PD2+PD3+PD4) of quantities of lights incident on each light receiving units, a difference {(PD1+PD2}−(PD3+PD4)} between optical signals received by the light receiving units arranged on the upper surface and optical signals received by the light receiving units arranged on the bottom surface, a difference {(PD1+PD3}−(PD2+PD4)} between optical signals received by the light receiving units arranged on the left surface and optical signals received by the light receiving units arranged on the right surface, and a difference {(PD1+PD4}−(PD2+PD3)} between optical signals received by the light receiving units arranged on a diagonal line, it can be determined whether all the heart rate sensor units are properly attached to the skin, or whether any one of the light receiving units is not attached to the skin.

Furthermore, a difference {(PD1+PD2}−(PD3+PD4)} between quantity of lights incident on the light receiving units (120, 130) arranged on the upper and quantity of lights incident on the light receiving units (140,150) arranged on the bottom surface is defined as a first error (E1), a difference {(PD1+PD3}−(PD2+PD4)} between quantity of lights incident on the light receiving units (120, 140) arranged on the left surface and quantity of lights incident on the light receiving units (130,150) arranged on the right surface is defined as a second error (E2), and a difference {(PD1+PD4}−(PD2+PD3)} among sums of quantities of lights incident on the light receiving units (120, 150, 130, 140) each arranged on a diagonal line is defined as a third error (E3).

Thus, when a lengthwise direction of the light source unit at the heart rate sensor unit is assumed as X axis, the first error (E1) may be a Y axis rotation error, the second error (E2) may be an X axis rotation error and the third error (E3) may be a Z axis rotation error. Furthermore, the first, second and third errors (E1, E2, E3) may be respectively a pitch error, a roll error and a yaw error.

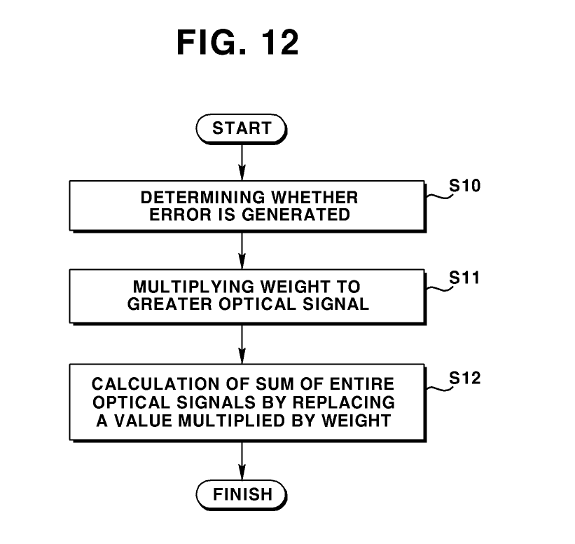

FIG. 12 is a schematic view illustrating a method for error compensation according to an exemplary embodiment of the present disclosure.

Referring to FIG. 12, the method for error compensation according to an exemplary embodiment of the present disclosure may include determining whether an error is generated (S10). At this time, the determination of whether an error is generated means, as explained in FIG. 11, whether the first, second and third errors are generated, whether the determination may be generation of any one error from the first, second and third errors, and may be generation of all errors. An ideal case would be there is no generation of errors.

The case of error generation means generation of errors when intensities of optical signals received by the first to fourth light receiving units (120, 130, 140, 150) are mutually compared, and in this case, a predetermined weight is multiplied to a greatest optical signal (S11). Then, a sum of values, where the weight-multiplied optical signal and remaining optical signals are all added up, is called a reference optical signal (S12).

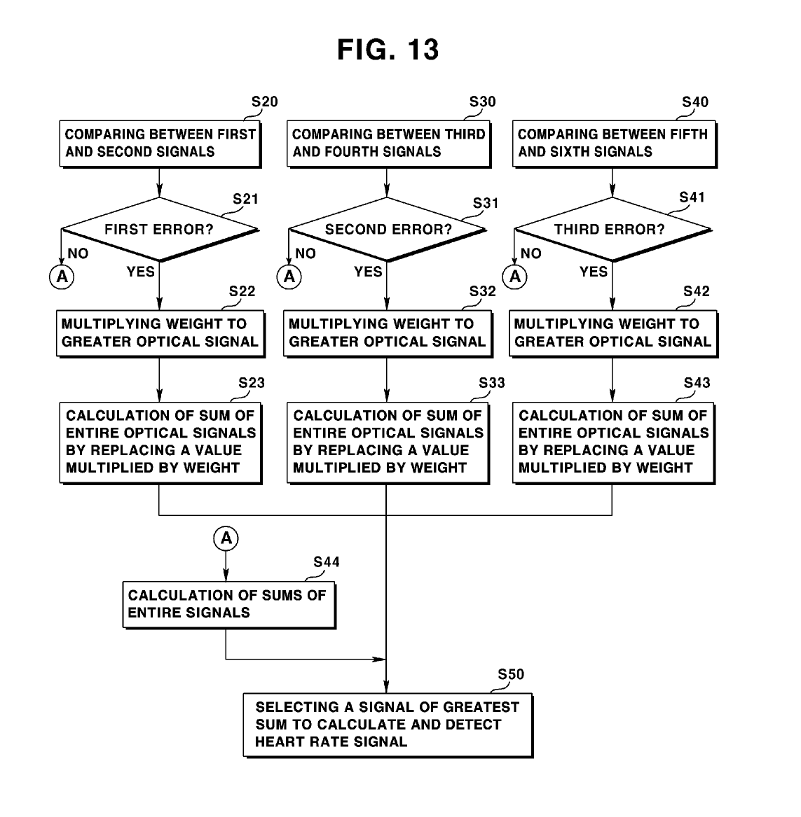

FIG. 13 is a schematic view illustrating a comparative signal weighting method in a method for error compensation according to an exemplary embodiment of the present disclosure.

Referring to FIG. 13, alignment errors that may be generated from the heart rate sensor applied by the method for error compensation according to an exemplary embodiment of the present disclosure may be the first, second and third errors respectively. For error compensation, first to sixth signals may be employed.

The first signal (PD1+PD2) is a sum of optical signals received by the light receiving units (120, 130) arranged on the upper surface. The second signal (PD3+PD4) is a sum of optical signals received by the light receiving units (140, 150) arranged on the bottom surface. The third signal (PD1+PD3) is a sum of optical signals received by the light receiving units (120, 140) arranged on the left surface. The fourth signal (PD2+PD4) is a sum of optical signals received by the light receiving units (130, 150) arranged on the right surface. The fifth signal (PD1+PD4) and the sixth signal (PD2+PD3) are sums of optical signals received by the light receiving units (120, 130, 140, 150) arranged on diagonal lines.

Therefore, the first error is a difference between the first and second signals. The second error is a difference between the third and fourth signals. The third error is a difference between the fifth and sixth signals. The first to third errors may be simultaneously generated, and any one error may be generated. An ideal case would be where all the optical signals received by the four light receiving units are same not to generate any error. Furthermore, a difference less than a predetermined value may be set up as a threshold value which is regarded as there being generated no error, and only a case where an error is greater than the threshold value may be determined as generation of error.

Now, the method for error compensation according to an exemplary embodiment of the present disclosure will be described in details.

First, the first and second signals are compared (S20). A determination is made as to whether the first error is generated through the comparison (S21).

The determination of generation of first error is to determine whether there is a difference between the first signal and the second signal, where the difference may be a positive value or a negative value. The determination of whether the difference is a positive value or a negative value can learn which signal between the first signal and the second signal is a greater value. Thus, a weight can be multiplied to the greater signal between the two signals (S22). The weight is a predetermined value, and when a calculation is made by multiplying the weight to an optical signal detected with the greatest signal, the most accurate heart rate measurement reference can be obtained. After the weight is multiplied, a new value multiplied with the weight and remaining optical signal not multiplied by the weight are added to obtain a sum of weighted signal (S23). The second error and the third error can be obtained by the abovementioned method.

The third signal and the fourth signal are compared (S30). Determination is made if the second error is generated through a result of the comparison (S30). The determination of generation of second error is to determine whether there is a difference between the third signal and the fourth signal, where the difference may be a positive value or a negative value. The determination of whether the difference is a positive value or a negative value can learn which signal between the third signal and the fourth signal is a greater value. Thus, a weight can be multiplied to the greater signal between the two signals (S32). After the weight is multiplied, a new value multiplied with the weight and remaining optical signal not multiplied by the weight are added to obtain a sum of weighted signal (S33).

The fifth signal and the sixth signal are compared (S40). Determination is made if the third error is generated through a result of the comparison (S40). The determination of generation of third error is to determine whether there is a difference between the third signal and the fourth signal, where the difference may be a positive value or a negative value. The determination of whether the difference is a positive value or a negative value can learn which signal between the third signal and the fourth signal is a greater value. Thus, a weight can be multiplied to the greater signal between the two signals (S42). After the weight is multiplied, a new value multiplied with the weight and remaining optical signal not multiplied by the weight are added to obtain a sum of weighted signal (S43).

Meanwhile, when the first, second and third errors are not generated, a sum of an entire signal is calculated (S44). The greatest value is selected to calculate and detect a heart rate signal after sums of weighted signals are obtained in response to whether each error is generated through the abovementioned methods.

A simpler explanation may be provided by way of example according to the following manner.

For example, if is assumed that the first received light signal (PD1) is 10, the second received light signal (PD2) is 20, the third received light signal (PD3) is 30 and the fourth received light signal (PD4) is 40, the first error is −40{(PD1+PD2}−(PD3+PD4)}, the second error is −20{(PD1+PD3}−(PD2+PD4)} and the third error is 0{(PD1+PD4}−(PD2+PD3)}.

The second signal (PD3+PD4) is greater in the first error, the fourth signal (PD2+PD4)} is greater in the second error. If the predetermined weight is 2, a sum of weighted signals according to the first error is 170{(PD1+PD2+(PD3*2)+(PD4*2)}, and a sum of weighted signals according to the second error is 160{(PD1+PD2*2)+PD3+(PD4*2)}. Thus, calculation and detection of heart rate signal may be obtained using 170 which is the greatest value among the sums of weighted signals according to each error.

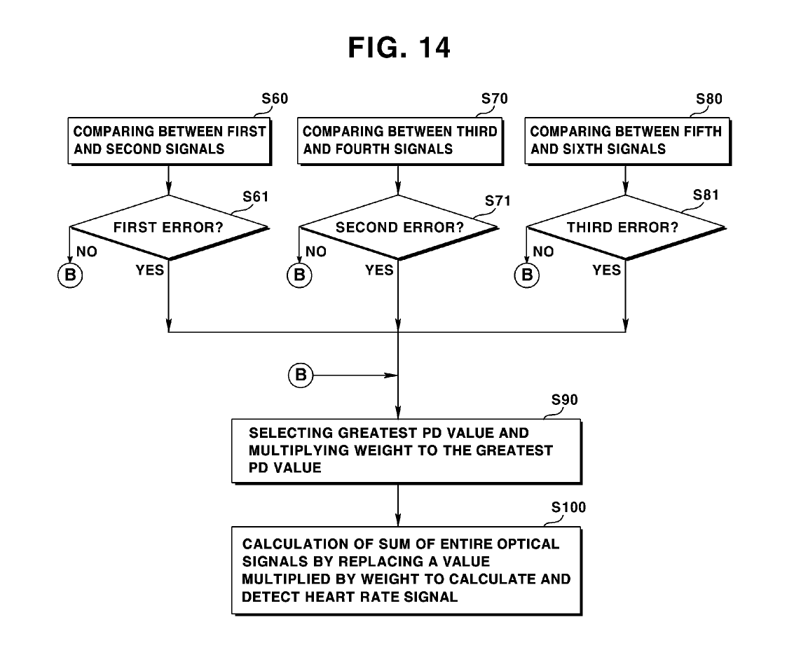

FIG. 14 is a schematic view illustrating an absolute signal weighting method according to an exemplary embodiment of the present disclosure.

Referring to FIG. 14, the absolute signal weighting method according to a method for error compensation according to an exemplary embodiment of the present disclosure may go through the following steps.

First, the first and second signals are compared (S60). A determination is made as to whether the first error is generated through the comparison (S61). The determination of generation of first error is to determine whether there is a difference between the first signal and the second signal. Successively, the third signal and the fourth signal are compared (S70). Determination is made if the second error is generated through a result of the comparison (S71). The determination of generation of second error is to determine whether there is a difference between the third signal and the fourth signal.

Next, the fifth signal and the sixth signal are compared (S80). Determination is made if the third error is generated through a result of the comparison (S81). The determination of generation of third error is to determine whether there is a difference between the third signal and the fourth signal.

As in the comparative signal weighted method in FIG. 13, the first to third errors may be simultaneously generated, or any one error may be generated. An ideal case may be where all the optical signals received by the four light receiving units are same not to generate any error. Furthermore, a difference less than a predetermined value may be set up as a threshold value which is regarded as there being generated no error, and only a case where an error is greater than the threshold value may be determined as generation of error.

When the first to third errors are detected, it can be learned which optical signal received by a light receiving unit among the first to four light receiving units (120, 130, 140, 150) is the greatest, in consideration of symbols and sizes of values thereof

Successively, an optical signal having the greatest value is selected and a weight is multiplied to the optical signal having the greatest value. Thereafter, the value multiplied with the weight and the remaining optical signals may be added (S100) to calculate and detect the heart rate signal.

A simpler explanation may be provided by way of example according to the following manner.

For example, if is assumed that the first received light signal (PD1) is 10, the second received light signal (PD2) is 20, the third received light signal (PD3) is 30 and the fourth received light signal (PD4) is 40, the first error is −40{(PD1+PD2}−(PD3+PD4)}, the second error is −20{(PD1+PD3}−(PD2+PD4)} and the third error is 0{(PD1+PD4}−(PD2+PD3)}.

When the first to third errors are determined, the fourth light receiving signal (PD4) having the greatest value can be obtained, and when weight of ‘2’ is multiplied to the fourth light receiving signal (PD4), and when a sum of new weights is obtained, which is 140{PD1+PD2+PD3+(PD4*2)}, by which the heart rate signal can be calculated and detected.

The method for error compensation of bio-information according to the present invention can choose an appropriate method from a comparative signal weighting method of FIG. 13 or an absolute signal weighting method of FIG. 14 in consideration of weight setting, SNR (Signal-to-Noise-Ratio) and operation environment of heart rate sensor.

As the present features may be embodied in several forms without departing from the characteristics thereof, it should also be understood that the above-described embodiments are not limited by any of the details of the foregoing description, unless otherwise specified, but rather should be considered broadly within its scope as defined in the appended claims, and therefore all changes and modifications that fall within the metes and bounds of the claims, or equivalents of such metes and bounds are therefore intended to be embraced by the appended claims.

What is claimed is:

1. An apparatus for measuring bio-information, the apparatus comprising:
a heart rate sensor unit configured to measure heart rates by receiving a light that has entered and come out from skin;
an acceleration sensor unit configured to output a step count by measuring the step count of a wearer;
a display unit configured to display the measured heart rates and step count; and
a wrist-wearable connection unit configured to electrically connect the heart rate sensor, the acceleration sensor unit and the display unit.

2. The apparatus of claim 1, wherein the heart rate sensor unit includes,
a light source unit configured to emit a linear light source,
a light receiving unit configured to receive the light that has entered and come out from the skin from the light emitted from the light source, and
a controller configured to detect the heart rates from a quantity of light received by the light receiving unit.

3. The apparatus of claim 2, wherein the light source unit includes,
an LED (Light Emitting Diode) configured to emit a light of point light source,
a curved light guide of a particular curvature radius configured to advance the emitted light to a particular direction,
a plurality of V-shaped patterns configured to emit a light to a particular direction by refracting the emitted light, and
a reflective plate configured to reflect a light emitted to an outside from the light guide into an interior of the light guide.

4. The apparatus of claim 3, wherein the LED uses a yellowish green color light source.

5. The apparatus of claim 2, wherein the light receiving unit includes,
a first light receiving unit arranged at an upper left surface based on a lengthwise direction of the light source,
a second light receiving unit arranged at an upper right surface based on a lengthwise direction of the light source,
a third light receiving unit arranged at a bottom left surface based on a lengthwise direction of the light source, and
a fourth light receiving unit arranged at a bottom right surface based on a lengthwise direction of the light source.

6. The apparatus of claim 2, wherein the light receiving unit includes,
a photodetector configured to receive a light that has entered and come out of skin from a light emitted from the light source unit, and
a light receiving house configured to wrap the photodetector and to gradually broaden at an entrance toward a skin contact surface.

7. The apparatus of claim 3, wherein the plurality of V-shaped patterns tapers off at a spacing of adjacent patterns as being distanced from the LED.

8. The apparatus of claim 1, wherein the connection unit is a wrist band type connector unit of elastic material.

9. The apparatus of claim 1, wherein the connection unit is a wrist watch type, wrist-attachable connector unit.

10. A method for error compensation in a heart rate sensor including a first light receiving unit arranged at an upper left surface of a line light source, a second light receiving unit arranged at an upper right surface of the line light source,
a third light receiving unit arranged at a bottom left surface of the line light source, and a fourth light receiving unit arranged at a bottom right surface of the line light source, the method comprising:
detecting an error by comparing light signals received by the first to fourth light receiving units;
multiplying a predetermined weight to a greater light signal as a result of comparison of the light signals when the error is generated; and
adding a size of a light signal multiplied by the weight to a size of a light signal not multiplied by the weight.

11. The method of claim 10, wherein the error includes a first error, which is a difference between a first signal, which is a sum of light signals received by the first and second light receiving units and a second signal, which is a sum of light signals received by the third and fourth light receiving units,
a second error, which is a difference between a third signal, which is a sum of light signals received by the first and third light receiving units and a fourth signal, which is a sum of light signals received by the second and fourth light receiving units, and
a third error, which is a difference between a fifth signal, which is a sum of light signals received by the first and fourth light receiving units and a sixth signal, which is a sum of light signals received by the second and third light receiving units.

12. The method of claim 11, wherein the step of multiplying a predetermined weight to a greater light signal as a result of comparison of the light signals when the error is generated includes,
multiplying a predetermined weight to a greater signal between the first and second signals when the first error is generated,
multiplying a predetermined weight to a greater signal between the third and fourth signals when the second error is generated, and
multiplying a predetermined weight to a greater signal between the fifth and sixth signals when the first error is generated.

13. The method of claim 12, wherein the method further comprises determining a greater value as a heart rate reference signal between a size of an optical signal multiplied by the weight and a size of an optical signal not multiplied by the weight.

14. A method for error compensation in a heart rate sensor including a first light receiving unit arranged at an upper left surface of a line light source, a second light receiving unit arranged at an upper right surface of the line light source,
a third light receiving unit arranged at a bottom left surface of the line light source, and a fourth light receiving unit arranged at a bottom right surface of the line light source, the method comprising:
detecting an error by comparing light signals received by the first to fourth light receiving units;
multiplying a predetermined weight to a greater light signal as a result of comparison of the light signals when the error is generated; and
adding a size of a light signal multiplied by the weight to a size of a light signal not multiplied by the weight.

15. The method of claim 14, wherein the step of detecting an error by comparing light signals received by the first to fourth light receiving units includes determining a light receiving unit having received a largest size of optical signal by comparing a first error, which is a difference between a first signal, which is a sum of light signals received by the first and second light receiving units and a second signal, which is a sum of light signals received by the third and fourth light receiving units, a second error, which is a difference between a third signal, which is a sum of light signals received by the first and third light receiving units and a fourth signal, which is a sum of light signals received by the second and fourth light receiving units, and a third error, which is a difference between a fifth signal, which is a sum of light signals received by the first and fourth light receiving units and a sixth signal, which is a sum of light signals received by the second and third light receiving units.
